(19) United States
(12) Patent Application Publication
(10) Pub. No.: US 2026/0061116 A1
(43) Pub. Date: Mar. 5, 2026

(54) IV DRIP CHAMBER FOR REDUCING TURBULENCE

(71) Applicant: Damea Alexander, Marysville, OH (US)

(72) Inventor: Damea Alexander, Marysville, OH (US)

(21) Appl. No.: 18/824,008

(22) Filed: Sep. 4, 2024

(51) Int. Cl. A61M 5/14; A61M 5/142

(52) U.S. Cl. CPC A61M 5/1411; A61M 5/142

(57) ABSTRACT

An intravenous (“IV”) drip chamber for reducing turbulence is disclosed, along with related systems and methods. The IV drip chamber includes an enclosure having an entrance and an exit. A ramp, such as with an at least substantially continuous incline, extends from a first location proximate the entrance, such as below the entrance, to a second location proximate the exit, such as above the exit. In this way, droplets of medication or other fluid may travel smoothly and/or with a smaller fall through the drip chamber, thereby reducing formation of micro-bubbles or other turbulence.

### CROSS-REFERENCE TO RELATED APPLICATIONS

This application is filed as original and therefore makes no priority claim.

### TECHNICAL FIELD

Exemplary embodiments relate generally to an IV drip chamber for reducing turbulence, as well as systems and methods related to the same.

### BACKGROUND AND SUMMARY OF THE INVENTION

Intravenous (IV) dispensing of medications and other fluids is known. IV systems generally include a drip chamber, sometimes also referred to as a drip bulb, that allows solution from a bag, bottle, or other container to drip into the drip chamber before entering IV tubing connected to a patient. This allows any gas in the fluid to rise out from the fluid so that it is not passed downstream, which can cause medical complications.

Certain medications, such as but not necessarily limited to plasma-derived medications used in transfusions, can be agitated by even the relatively small droplets of fluid falling over the relatively small distance within a drip chamber. This agitation can create micro-bubbles within the fluid or other turbulence. While sometimes resolved within the drip chamber, these micro-bubbles or other turbulence sometimes persist in the fluid and are carried therewith beyond the drip chamber. The persistence of such micro-bubbles or other turbulence can cause connected equipment to malfunction or alert. For example, connected IV pumps, which may be fluidly connected to and/or forming part of the IV system, may alarm due to the persistence of such micro-bubbles or other turbulence, which is a nuisance for patients and providers, and correction of which takes time away from more productive activities like other patient care.

What is needed is an IV system which reduces micro-bubble and/or other turbulence formation. An IV drip chamber for reducing micro-bubble and/or other turbulence formation is provided, along with related systems and methods.

An IV drip chamber is provided which includes a ramped surface. The ramp begins near a fluid entrance such that drips of the solution are captured by the ramp and passed down the same to a location proximate a fluid exit for the drip chamber. The ramp may be at least substantially linear (e.g., along a longitudinal axis thereof), have a helical shape, and/or take on other forms. The ramp may be inclined in an at least substantially continuous fashion, such as with an at least substantially constant slope. Optionally, the ramp may include one or more features (e.g., material selection, surface textures, tapered surfaces, combinations thereof, or the like) that facilitate droplet travel. In this way, the drips into the IV chamber can be passed gently into the pooled fluid at a bottom of the drip chamber, thereby preserving the gas removal feature while reducing or eliminating a drop distance of the droplets, thereby reducing or eliminating the formation of micro-bubble or other turbulence. Optionally, a mesh may be provided within the drip chamber, such as at or below an exit from the ramp, to further arrest movement, prevent splashing, and the like to inhibit the formation of micro-bubble or other turbulence.

Further features and advantages of the systems and methods disclosed herein, as well as the structure and operation of various aspects of the present disclosure, are described in detail below with reference to the accompanying figures.

### BRIEF DESCRIPTION OF THE DRAWINGS

In addition to the features mentioned above, other aspects of the present invention will be readily apparent from the following descriptions of the drawings and exemplary embodiments, wherein like reference numerals across the several views refer to identical, similar, or equivalent features, and wherein:

FIG. 1 is front view of an exemplary prior art IV drip chamber;

FIG. 2 is a front view of an exemplary IV drip chamber for reducing turbulence;

FIG. 3 is a detailed top view of a ramp within the IV drip chamber, taken at detail A of FIG. 2;

FIG. 4 is a perspective view of another exemplary IV drip chamber for reducing turbulence;

FIG. 5 is a detailed perspective view of the IV drip chamber, taken at detail B of FIG. 4;

FIG. 6 is a front view of the IV drip chamber of FIG. 4;

FIG. 7 is a plan view of an exemplary system including the IV drip chamber of FIG. 1-6; and

FIG. 8 is a flow chart with an exemplary method for installing and operating the system of FIG. 7.

### DETAILED DESCRIPTION OF EXEMPLARY EMBODIMENT(S)

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein can be made without departing from the scope and spirit of the present invention. In addition, descriptions of well-known functions and constructions are omitted for clarity and conciseness.

Embodiments of the invention are described herein with reference to illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

FIG. 1 illustrates an exemplary known IV drip chamber 10. Droplets 16 of a fluid, such as a medication, may drip from an entrance 14 into, and through at least part of, an enclosure 12, such as into a pool 18 of the fluid located adjacent to an exit 20 from the enclosure 12. The droplets 16 may fall a distance, and when doing so, may create micro-bubbles or other turbulence within the pooled fluid 18, thereby resulting in downstream micro-bubbles or other turbulence.

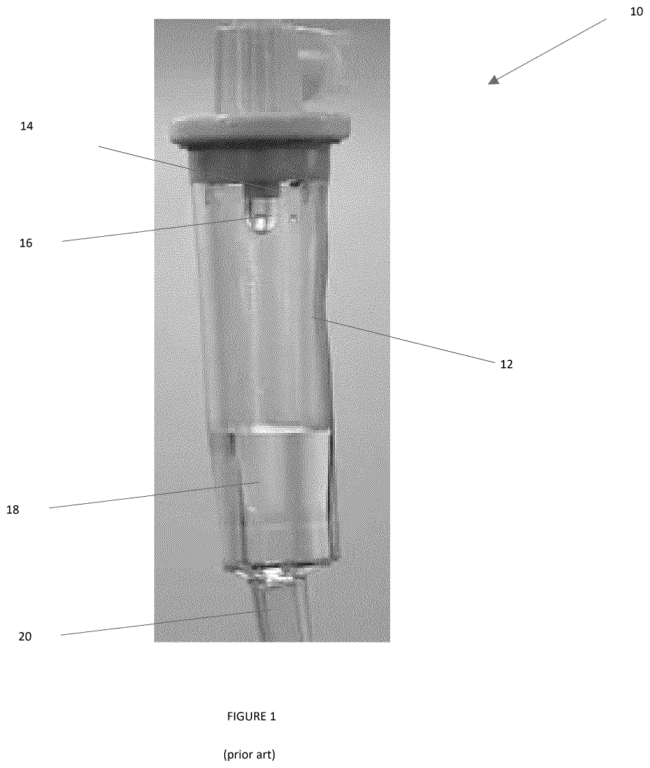

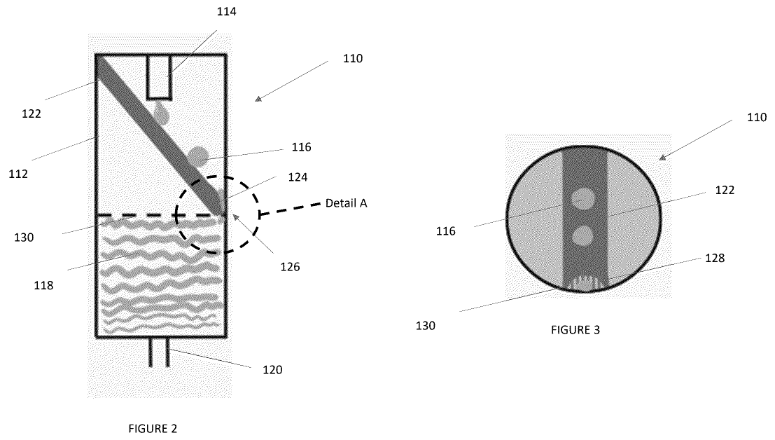

FIG. 2 and FIG. 3 illustrate an exemplary IV drip chamber 110 for reducing turbulence. Similar items may be numbered similarly but increased by 100 (e.g., 10 to 110, 12 to 112, etc.). A ramp 122 may be provided within an enclosure 112. In exemplary embodiments, without limitation, the ramp 122 extends below an entrance 114 into the enclosure 112 and may terminate above an exit 114 thereof, such as but not limited to adjacent to an anticipated fluid line for pooled fluid 118 within the enclosure 112 when the IV drip chamber 110 is in normal use. The ramp 122 may act as a slide for the droplets 116 and/or a surface for the droplets 116 to roll or otherwise travel down. The ramp 122 may extend between the entrance and exit 114, 120 and, in exemplary embodiments, without limitation, have one or more of: a linear, or at least substantially (e.g., within 10%) linear longitudinal axis (essentially, extends in a straight-line), a constant, or at least substantially (e.g., within 10%) constant slope, a continuous, or at least substantially (e.g., within 10%) continuous incline, and a flat, or at least substantially (e.g., within 10%) flat upper surface. The anticipated fluid line for pooled fluid 118 may be at a lower half, or lower quarter, of the enclosure 112, though such is not required. The pooled fluid 118 may accumulate adjacent to the exit 114 for the enclosure 112, which may be fluidly connected to IV tubing or other parts of the IV system. The ramp 122 may extend various distances, such as above, at, or below the anticipated fluid line for the pooled fluid 118 within the enclosure 112.

The ramp 122 may be connected at a proximal end thereof to an upper portion of the enclosure 112, such as at an upper portion of a sidewall thereof and/or at an upper surface thereof. The ramp 122 may extend, such as at a constant or at least substantially (e.g., within 10%) constant downward angle, across the enclosure 112, such as along a diameter thereof, to an upper portion of the enclosure 112, such as at a lower portion of the sidewall thereof and/or at the lower surface thereof. In exemplary embodiments, without limitation, the ramp 122 extends from an upper half of the enclosure 112, and more preferably an upper quarter thereof, to a lower half of the enclosure 112, and more preferably a lower quarter thereof.

The ramp 122 may terminate a small distance from the sidewall of the enclosure 112 such that a gap 126 is formed between at least part of a distal end of the ramp 122 and an adjacent portion of the sidewall of the enclosure 112. In this way, a distal end of the ramp 122 may be otherwise unattached to the enclosure 112 (e.g., be a free end). In exemplary embodiments, the gap 126 is 2-4 mm, though other size and/or shape gaps 126 may be provided.

Alternatively, or additionally, the ramp 122 may connect to the sidewall of the enclosure 112 and may include an aperture 128 in the ramp 122 for the droplet 116 to travel through and into the pooled fluid 118. In this way, the ramp 122 may be connected to the enclosure 112 at both ends thereof. The aperture 128 may be a single aperture or multiple apertures, and may be of various size and/or shape, such as but not limited to, oval, circular, half circle, slots, combinations thereof, or the like.

The ramp 122 may be separately formed and connected to the enclosure 112 by adhesive, welding, combinations thereof, or the like. Alternatively, or additionally, the ramp 122 may be integrally formed with the enclosure 112.

The ramp 122 may comprise a flat, or substantially (e.g., within 20%) flat, upper surface, and/or may be cupped, such as in a “U” shape, to contain the droplets 116 therein.

The ramp 122 may occupy some or all of the lateral cross-section of the enclosure 112. In exemplary embodiments, the ramp 112 is smaller in lateral dimension than a diameter or other lateral dimension of the enclosure 112, such as illustrated with particular regard to FIG. 3. For example, without limitation, the ramp 112 may take on a generally cuboid shape. However, other size and/or shape ramps 122 may be utilized. A centerline of the ramp 122, in exemplary embodiments, without limitation, may extend along, or at least substantially along (e.g., within 10%) a diameter of an upper and lower surface of the enclosure 112.

In exemplary embodiments, without limitation, a mesh 130 may be provided within the enclosure 112. Inclusion of the mesh 130 is optional. The mesh 130 being preferably located at or below a second, distal end of the ramp 122, such as at the aperture 128, between the ramp 122 and the sidewall and/or the exit 120 from the enclosure 112. In exemplary embodiments, without limitation, the mesh 130 may extend from a distal end of the ramp 122 to the sidewall of the enclosure 112. The mesh 130 may cover all, or at least substantially (e.g., 90% or more) all the aperture 128 and/or the gap 126 by way of non-limiting example. The mesh 130 may extend below all, or at least substantially (e.g., 90% or more) all of the distal end of the ramp 122, the aperture 128, and/or the gap 126 by way of non-limiting example. The mesh 130 may act to further arrest and/or slow the droplets 116 and/or disperse the droplets 116 into smaller droplets 116 before entering the pooled fluid 118 or the exit 120. Alternatively, or additionally, the mesh 130 may act to prevent splashing. Regardless, the mesh 130 may further reduce micro-bubble formation or other turbulence. The mesh 130 may be attached at one or more locations to the sidewall of the enclosure 112.

Alternatively, or additionally, the mesh 130 (or a second mesh) may be configured to float atop the pooled fluid 118, otherwise freely rest within the enclosure 112 (e.g., be otherwise unattached therefrom), and/or be provided at another part of the enclosure 112, such as separate from the ramp 122 (attached or free-floating, unattached). The mesh(es) 130 may occupy some, all, or substantially (e.g., at least 90%) of a lateral cross section of the enclosure 112. For example, without limitation, the mesh(es) 130 may comprise a diameter matching, or at least substantially matching (e.g., within 10%) a diameter of the enclosure 112. However, the mesh(es) 130 may be of various sizes and/or shapes. The mesh(es) 130 are optional.

While the enclosure 112 may be sometimes shown and/or described as being cylindrical in shape, other size and/or shape enclosures 112 may be utilized.

The entrance 114 and/or exit 120 may comprise apertures located in the enclosure 112 and/or tubular or other shaped structure which extend into and/or outside of the enclosure 112.

In exemplary embodiments, without limitation, an upper surface of the ramp 122 extends within 7-10 mm of the entrance 114 and within 7-10 mm of the exit 120, by way of non-limiting example.

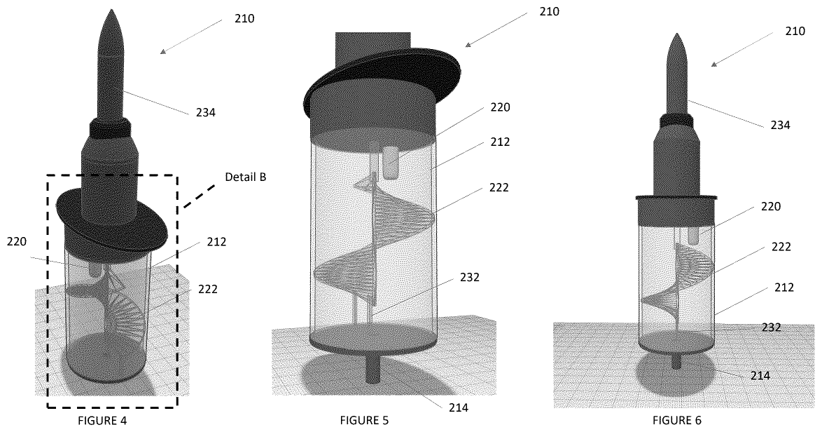

FIG. 4 through FIG. 5 illustrate another exemplary embodiment of the IV drip chamber 210 for reducing turbulence. Similar items may be numbered similarly but increased by 100 (e.g., 10 to 110 or 210, 12 to 112 or 212, etc.).

A helical shaped ramp 222 may be provided within an enclosure 212 of the drip chamber 210. The enclosure 212 may be cylindrical in shape. The helical shaped surface of the ramp 222 may extend about a support shaft 232, which may extend between upper and lower surfaces of the enclosure 212. The support shaft 232 may be attached to the upper and lower surfaces of the enclosure 212, such as by adhesive, friction fit (e.g., ends of support shaft 232 each fit within a respective aperture in the respective surface), welding, combinations thereof, or the like, and/or integrally formed with the same. The ramp 222 surface may extend along, adjacent to, and/or be attached to, a sidewall of the enclosure 212. In exemplary embodiments, without limitation, the ramp 222 is integrally formed with at least the sidewall portion of the enclosure 212. The helical arrangement may enhance ease of manufacture, stability, and/or droplet capture, among other advantages.

A first end of the ramp 222 may be located proximate to an entrance 214 to the enclosure 212. A second end of the ramp 222 may be located proximate to an exit 220 of the enclosure 212. The ramp 222 may extend various distances, such as above, at, or below the anticipated fluid line for the pooled fluid within the enclosure 212.

At least a majority of an upper surface of the ramp 112, 222 may comprise a flat, smooth, and/or substantially (e.g., within 10%) flat and/or smooth surface for droplet travel. Optionally, the ramp 122, 222 may include one or more features (e.g., material selection, surface textures or features, tapered surfaces, combinations thereof, or the like) that facilitate droplet travel. For example, without limitation, at least a majority of an upper surface of the ramp 112, 222 may comprise one or more polymers, may be coated with one or more non-stick materials, and/or have one or more longitudinally extending channels or protrusions which facilitate droplet travel.

An IV spike 234 may be connected to, and/or integrally formed with, the enclosure 212, and therefore may optionally form part or, or be connected with, the drip chamber 210.

The ramp 122, 222 may be provided in various sizes, shapes, and/or arrangements within the enclosure 112, 212. Various size, shape, and/or type of IV drip chambers 110, 210, such as with various size, shape, and/or location entrances 114, 214 and/or exits 120, 220 may be utilized, such as with various size, shape, and/or type of enclosures 112, 212, and in such embodiments, the ramp(s) 122, 222 may be adapted accordingly (e.g., to extend from a first location gravitationally proximate the entrance 114, 214 under normal use so as to catch droplets entering the drip chamber 110, 210, to a second location gravitationally proximate the exit 120, 220 under normal use so as to allow the droplets to exit the drip chamber 110, 210, preferably after first being gathered in a pool within the drip chamber 110, 210).

Multiple ramps 122, 222 may optionally be employed, such as in stages zig-zagging the enclosure 112, 212, extending along a sidewall of the enclosure 112, 212, or in other various configurations.

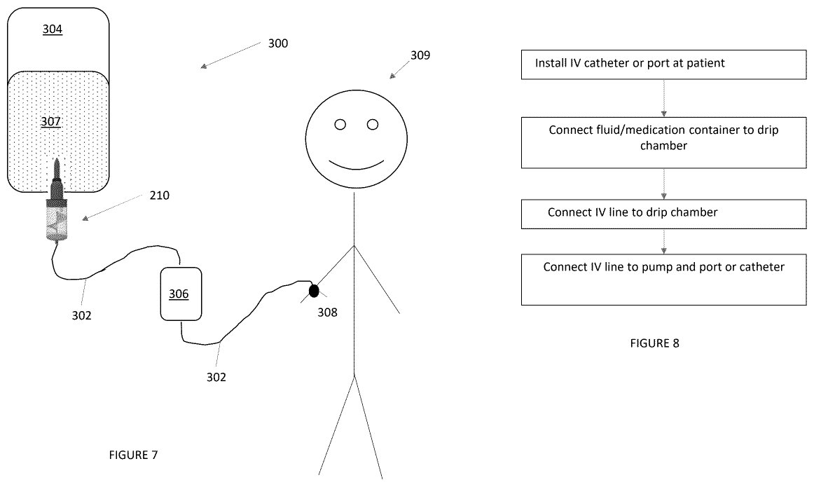

As illustrated with particular regard to FIG. 7 and FIG. 8, the IV drip chambers 110, 210 may be utilized as part of IV systems 300, and may be attached to and/or integrally formed with various components thereof (e.g., IV tubing 302, fluid/medication containers 304, IV pumps 306, IV spikes 234, IV ports and/or catheters 308, combinations thereof, or the like) and may be utilized as part of such IV systems 300 in various ways to dispense medications, perform transfusions, combinations thereof, or the like.

In exemplary embodiments, without limitation, the IV drip chamber 110, 210 is fluidly connected to a container 304 comprising the medication or other fluid 307 for dispensing. The medication or other fluid 307 may travel from the container 304 through the entrance 114, 214 and into the enclosure 112, 212 of the IV drip chamber 112, 212. IV tubing 302 may be fluidly connected to the exit 120, 220 to the enclosure 112, 212 of the IV drip chamber 110, 210. The IV tubing 302 may be fluidly connected to a patient's 309 vascular system, such as at one or more veins thereof, such as by way of patient interface/connection components 308 (e.g., IV ports, catheters, needles, combinations thereof, or the like). An IV pump 306 may be connected to the IV tubing 302. The IV pump 306 may be operated to cause flow of medication 307 from the container 304 into the patient's 309 vascular system. These exemplary steps may be provided in different order, and some or all of the steps may be repeated or omitted.

While intravenous applications are sometimes shown and/or discussed, the disclosed drip chamber 110, 210 may be utilized in other applications, such as with other types or kinds of fluids, such as with regard to various industrial fluids and/or applications.

Any embodiment of the present invention may include any of the features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention.

What is claimed is:

1. An intravenous (“IV”) drip chamber for reducing turbulence, said IV drip chamber comprising:
an enclosure having an entrance at an upper portion of the enclosure and an exit at a lower portion of the enclosure; and
a ramp extending from a first location adjacent to the entrance to a second location adjacent to the exit.

2. The IV drip chamber of claim 1 wherein:
the entrance is located at, or extends to, an upper surface of the enclosure;
the exit is located at, or extends to, a lower surface of the enclosure;
the first location for the ramp is below the entrance; and
the second location for the ramp is above the exit.

3. The IV drip chamber of claim 2 wherein:
the ramp comprises at an at least substantially continuous incline.

4. The IV drip chamber of claim 3 wherein:
the ramp is attached to one or both of a sidewall and the upper surface of the enclosure at a first end thereof.

5. The IV drip chamber of claim 4 wherein:
the ramp has an at least substantially linear longitudinal axis.

6. The IV drip chamber of claim 5 wherein:
the ramp has an at least substantially constant slope.

7. The IV drip chamber of claim 6 wherein:
the ramp is free at a second end thereof.

8. The IV drip chamber of claim 6 wherein:
the ramp is attached to the sidewall of the enclosure at a second end thereof;
an aperture is provided at the second end of the ramp.

9. The IV drip chamber of claim 8 further comprising:
a mesh provided at the aperture.

10. The IV drip chamber of claim 6 wherein:
at least a majority of an upper surface of the ramp is at least substantially flat.

11. The IV drip chamber of claim 6 wherein:
at least a majority of an upper surface of the ramp has a cupped shape.

12. The IV drip chamber of claim 6 further comprising:
a mesh provided below the ramp and spanning at least substantially all of a lateral cross section of the enclosure.

13. The IV drip chamber of claim 3 wherein:
the ramp forms a helical shape.

14. The IV drip chamber of claim 13 further comprising:
a support shaft extending between the upper surface of the enclosure and the lower surface of the enclosure, wherein the ramp extends at least partially about the support shaft to form the helical shape.

15. The IV drip chamber of claim 14 further comprising:
a first aperture located in the lower surface of the enclosure which accommodates a first end of the support shaft; and
a second aperture located in in the upper surface of the enclosure which accommodates a second end of the support shaft.

16. The IV drip chamber of claim 13 wherein:
the ramp is attached to a sidewall of the enclosure.

17. The IV drip chamber of claim 2 wherein:
the ramp comprises multiple surfaces which are spaced apart from one another.

18. A system for intravenous (“IV”) delivery of medication, said system comprising:
an IV drip chamber comprising:
an enclosure having an entrance located at an upper surface of the enclosure and an exit located at a lower surface of the enclosure; and
a ramp having an upper surface forming an at least substantially continuously incline at an at least substantially constant slope extending from a first location below the entrance to a second location above the exit.

19. The system of claim 18 further comprising:
a container for medication fluidly connected to said entrance;
IV tubing fluidly connected to said exit; and
an IV pump located along the IV tubing.

20. A method for intravenous (“IV”) delivery of medication, said method comprising:
providing an IV drip chamber comprising:
an enclosure having an entrance located at an upper surface of the enclosure and an exit located at a lower surface of the enclosure; and
a ramp comprising an at least substantially continuous incline at an at least substantially constant slope extending from a first location below the entrance to a second location above the exit;
fluidly connecting an IV catheter or port to a patient's vascular system;
fluidly connecting a container with medication to said entrance to said enclosure of said IV drip chamber;
fluidly connecting IV tubing to said exit to said enclosure of said IV drip chamber;
fluidly connecting said IV tubing to said IV catheter or port;
connecting an IV pump to said tubing; and
operating said IV pump to cause flow of medication from said container into the patient's vascular system.
